(19) United States
(12) Patent Application Publication
(10) Pub. No.: US 2016/0082134 A1
(43) Pub. Date: Mar. 24, 2016

(54) Nanoparticle Photoacoustic Imaging Agents

(71) Applicant: THE TRUSTEES OF PRINCETON UNIVERSITY, Princeton, NJ (US)

(72) Inventors: Robert K. Prud'homme, Lawrenceville, NJ (US); Vikram J. Pansare, Bloomington, IN (US); Shahram Hejazi, Princeton, NJ (US); Hoang D. Lu, Woodside, NY (US)

(73) Assignee: THE TRUSTEES OF PRINCETON UNIVERSITY, Princeton, NJ (US)

(21) Appl. No.: 14/747,397

(22) Filed: Jun. 23, 2015

Related U.S. Application Data
(60) Provisional application No. 62/015,835, filed on Jun. 23, 2014.

(51) Int. Cl. A61K 49/22

(52) U.S. Cl. CPC A61K 49/222; A61K 49/221

(57) ABSTRACT

The invention described herein relates to colloidal particles useful for photoacoustic imaging. The particles comprise a photoacoustic imaging agent with an absorbance maximum or plateau in the range of wavelengths 700-1100 nm. The imaging agent also displays low optical absorbance at some wavelength in the range 700-1100 nm. This combination of high and low optical absorbance enables background subtraction in photoacoustic imaging applications. The imaging agent is an organic compound having low aqueous solubility so that it is stably encapsulated in the hydrophobic core of the particle. The particle is stabilized by a polymeric surface coating, and the polymeric stabilizing layer on the surface of the particle may contain targeting ligands for targeted diagnostics or therapeutic delivery. The particle core may also contain therapeutic agents or other imaging agents.

### CROSS-REFERENCE TO PRIOR FILED APPLICATIONS

This application claims the benefit of U.S. Provisional Application No. 62/015,835, filed Jun. 23, 2014, which is incorporated herein in its entirety.

### FIELD OF INVENTION

The present disclosure generally relates to imaging agents, and in particular to photoacoustic imaging agents.

### BACKGROUND

Photoacoustic imaging is an emerging method for obtaining optical contrast from biological tissues and detecting it with ultrasound imaging.[4] High frequency, pulsed laser light penetrates tissue, generating a thermoelastic expansion dependent on optical absorption at the excitation wavelength of the light. This expansion induces pressure (ultrasound) waves which can then be detected with an ultrasound transducer.

To better visualize a target during an imaging study, contrast agents are often introduced into the animal or human subject. These contrast agents are localized at the site of interest and have significantly different optical properties than tissue, which allows for deeper and clearer imaging of in vivo structures.

The most commonly used contrast agents for preclinical photoacoustic imaging are fluorescent dyes repurposed from optical imaging including indocyanine green (ICG), IRDye800CW, AlexaFluor 750, and methylene blue.[5, 6] These dyes are soluble in aqueous phases and go to regions of high blood flow. Their drawback is that they are fluorescent with a relatively high quantum yield, which means much of the absorbed energy is emitted as light rather that converted into a photoacoustic signal. Also, these soluble dyes must be convalently conjugated to targeting agents to target them for diagnostic applications.

Other untargeted contrast agent approaches reported in the patent literature include encapsulated alkali and alkali earth halides, [7] micro- and nano-bubbles, [8, 9] and oxides with a metal coating on the core. [10]

To improve localization of contrast agent at the site of interest, targeted contrast agents have been developed. These approaches have coupled a targeting agent, such as an antibody or RGD peptide, onto a carrier with desirable photoacoustic characteristics. For these targeting approaches, nanoparticle formats including gold nanorods,[11, 12] carbon nanotubes,[13] and iron oxide particles linked to targeting ligands[14] have proven most flexible to vary the targeting species and the photoacoustic imaging agent. However, attempts at introducing such agents have encountered problems. Gold nanorods must be relatively long to achieve long wavelength excitation, but these large particles have poor biodistribution qualities. Carbon nanotubes have a very long aspect ratio and there is considerable concern about mitochondrial damage by carbon nanotubes.

All of these existing targeted and contrast agents typically suffer from one or more issues in their depth of imaging, biocompatibility, biodistribution, reproducibility, or scalability—thus limiting their utility in photoacoustic imaging.[15] The development of robust contrast agents would greatly extend photoacoustic applications by enabling deeper imaging within tissue, better visualization through enhanced contrast, and cell-specific imaging via targeting ligands.

None of the contrast agents described in the literature combine the features of high loading of an organic photoacoustic imaging (PAI) agents into a nanoparticle, where the optical properties of the PAI agent include strong optical absorption in the wavelength range 700 nm to 1100 nm with low absorption occurring somewhere in this range of wavelengths, and where the nanoparticle may be targeted with simple conjugation to the steric polymer layer on the nanoparticle surface.

The importance of the combination of strong absorption in this wavelength window, and also minimal absorbance at some wavelength range within this window is that this enables background subtraction and enhanced image quality in the photoacoustic image processing. Since modern PAI instruments use tunable lasers the background image of subject can be taken at a wavelength where the dye does not absorb, and then the laser wavelength can be tuned to the absorption maximum to obtain the second image. The subtraction of these two images enable much higher resolution of the subject than can be obtained from a single PAI image.

The previously reported nanoparticle contrast agents do not satisfy this criteria of strong and weak adsorption in the wavelength range of 700 nm to 1100 nm. Examples of this are carbon nanotube contrast agents shown in FIG. 1 where the minimum in the absorbance is 0.35 at 850 nm and a maximum at 1100 nm of 0.55.[1] The difference between the high and low values is only 64%, and the minimum in absorbance is not within 15% of baseline with respect to the absorbance maximum. Similarly, gold nanorods show absorption maxima at wavelengths shorter than 700 nm (FIG. 2[2]) or show absorbance values that do not fall to within 15% of baseline within the range. Also, these contrast agents are often based on inorganic compounds, such as the gold nanorods presented in FIG. 2.

### SUMMARY OF THE INVENTION

Nanoparticle constructs for photoacoustic imaging are disclosed. The Nanoparticle includes a photoacoustic imaging agent (PAI) agent which has an absorbance maximum or plateau in the range of wavelengths 700-1100 nm, and which has a low absorbance value that is within 15% of baseline absorbance in this range. The PAI agent is an organic compound, The PAI agent has a solubility of less than 0.1 wt % in DI water at 20 C. The nanoparticle has a size range from 20 nm to 1500 nm. The nanoparticle has a core and a polymeric surface stabilizing layer. The PAI agent is encapsulated in the core of the nanoparticle.

The nanoparticles may be formed with sizes from 20 nm to 400 nm. The nanoparticles may include excipients or therapeutic agents. The nanoparticles may include an amphiphilic stabilizer. The nanoparticles may include targeting ligands on the stabilizer to enable targeting of the PAI nanoparticle. The nanoparticles may be formed by copolymer directed rapid precipitation. The nanoparticles may be formed by Flash NanoPrecipitation. The nanoparticles may be formed by emulsion processes followed by solvent stripping. The PAI agent may have a solubility of less than 0.05 wt % in DI water at 20 C. The PAI agent may have a solubility of less than 0.01 wt % in DI water at 20 C. The PAI agent may have a desired low solubility by virtue of ion pair formation. The PAI agent may have a desired low solubility by virtue of covalent conjugation. The PAI agent may be at least one of bacteriochlorin, chlorin, isobacteriochlorin and porphyrin. The PAI agent may be a hexacene-based dye. The PAI agent may include dyes available under the trade designations Lumogen IR 765 and 788. The PAI agent may include perylene and naphthyl dyes. The PAI agent may be a phthalocyanine based dye. The PAI agent may be a naphthalocyanine based dye. The PAI agent may be a dye available from Persis Science Inc. under the trade designation Par765, Par788, Par830, and Par900. The organic compound may include a coordinated metal atom. The nanoparticles may be useful for diagnostic imaging. The nanoparticles may be useful for animal imaging.

### BRIEF DESCRIPTION OF THE FIGURES

FIG. 1 is a graph showing the optical absorbance of carbon nanotube dispersions. The spectra shows broad absorbance and no region of baseline absorbance in the range 700-1100 nm.

FIG. 2 is a graph showing the absorbance for gold nanorods (GNR) with various surface functionalization as described in[2]. The absorption maximum is below 700 nm, rather than being in the range 700-1100 nm.

FIG. 3 is a graph showing the absorbance spectra for the bacteriochlorin series of PAI agents described in Taniguchi, et al. (2008).[3]

FIG. 4 is a graph showing the normalized absorbance spectrum for LW2.

FIG. 5 is a graph showing the normalized absorbance spectrum for IR122.

FIGS. 6A and 6B are graphs showing the unencapsulated absorbance spectra for BASF Lumogen 765 and 788 PAI agents.

FIGS. 7A-7D are graphs showing dynamic light scattering size distribution spectra of PAI nanoparticles formed using commercially available FIG. 7A—Par765, FIG. 7B—Par788, FIG. 7C—Par830, and FIG. 7D—Par900 dyes from Persis Science Inc.

FIGS. 8A-8D are graphs showing Normalized absorbance spectra of unencapsulated PAI dyes and encapsulated PAI dyes in nanoparticles using commercially available FIG. 8A—Par765, FIG. 8B—Par788, FIG. 8C—Par830, and FIG. 8D—Par900 dyes from Persis Science Inc. Par900 NPs absorb at a maximum value at 890 nm wavelength, and absorb 9.8% the maximum value at 1100 nm wavelength.

FIGS. 9A-9C are graphs showing varying sizes of PAI NPs including FIG. 9A—Dynamic light scattering size distribution spectra and FIG. 9B—intensity-weighted diameters of PAI nanoparticles formed using Par765 PAI at different compositions. FIG. 9C—Formulation summaries used to make Par765 NPs of varying sizes.

FIGS. 10A and 10B are graphs showing the formation of targeting PAI NPs. FIG. 10A—Dynamic light scattering size distribution spectra and FIG. 10B—absorbance spectra of Par series nanoparticles with or without folate targeting ligands.

FIG. 10C is table showing the formulation summaries used to make targeting Par series NPs of FIGS. 10A and 10B.

FIGS. 11A-11D are graphs showing the optical stability of targeting PAI NPs. FIGS. 11A-11D: Absorbance profiles of folic acid functionalized and non-functionalized control Par series NPs.

FIG. 11E is a table showing the formulation summaries used to make targeting Par series NPs of FIGS. 11A-11D.

FIG. 12 is a graph showing the photoacoustic activity of Par series NPs. Photoacoustic activity of Par788 or Par900 NPs was measured using a Vevo LAZR Photoacoustic Imaging System.

FIGS. 13A-13D are photos showing imaging of tumors using photoacoustic NPs. FIG. 13A—High resolution ultrasound-based visualization of the anatomy of a KB tumor implanted in a mouse. FIG. 13B—3D coregistered ultrasound/photoacoustic-based visualization of tumor anatomy & targeted nanoparticle distribution, after the mouse was injected with folate modified Par788 NPs. FIG. 13C—Spectrally unmixed photoacoustic image showing targeted nanoparticle distribution and blood signals. FIGS. 12A-13C are of the same mouse. FIG. 13D—Spectrally unmixed photoacoustic image showing non-targeted nanoparticle distribution and blood signals when non-targeted Par788 NPs were injected into a mouse implanted with a KB tumor.

FIGS. 14A-14C are photos showing simultaneously imaging of a tumor using two photoacoustic NPs at different wavelengths. FIG. 14A—Ultrasound image of a KB tumor implanted in a mouse with blood signals. FIG. 14B—Image of a KB tumor injected with folate targeting Par900 NPs, coregistered with Par900 NP, ultrasound, and blood signals. FIG. 14C—Image of a KB tumor injected with nontargeted Par788 NPs (after being injected with folate targeting Par900 NPs), coregistered with Par788 NP, Par900 NP, ultrasound, and blood signals.

FIG. 15A-15C are photos showing simultaneously imaging a tumor using two photoacoustic NPs at different wavelengths over time.

FIG. 15D is a graph showing spectrally unmixed nanoparticle signal quantification in a 3D tumor volume over time.

FIGS. 16A and 16B are graphs showing dynamic light scattering size distribution spectra of PAI nanoparticles formed using the commercially available B56 bacteriochlorin dye from NIRvana Sciences. With a FIG. 16A—1.8 kDa polystyrene homopolymer or FIG. 16B—alpha-tocopherol core and at varying % B56 load.

FIGS. 17A-17D are graphs showing the absorbance profiles of B56 NPs. FIG. 17A—Absorbance spectra of B56 polystyrene core NPs with varying core B56 loading (PS, B56%), and of unencapsulated B56 dissolved in THF (6% THF). FIG. 17B—Absorbance of B56 polystyrene core NPs at 720 nm with varying particle core mass loading (VitE, B56%). FIG. 17C—Absorbance spectra of B56 alpha-tocopherol core NPs with varying B56 loading, and of B56 dissolved in THF. FIG. 17D—Absorbance of B56 alpha-tocopherol core NPs at 720 nm with varying particle core mass loading.

FIG. 18 is a graph showing the normalized absorbance profiles of B56 PAI NPs. Normalized absorbance spectra of B56 polystyrene core NPs with 6% core B56 loading (PS, 6%), B56 alpha-tocopherol core NPs with 6% core B56 loading (VitE, 6%), and of unencapsulated B56 dissolved in THF (6% THF).

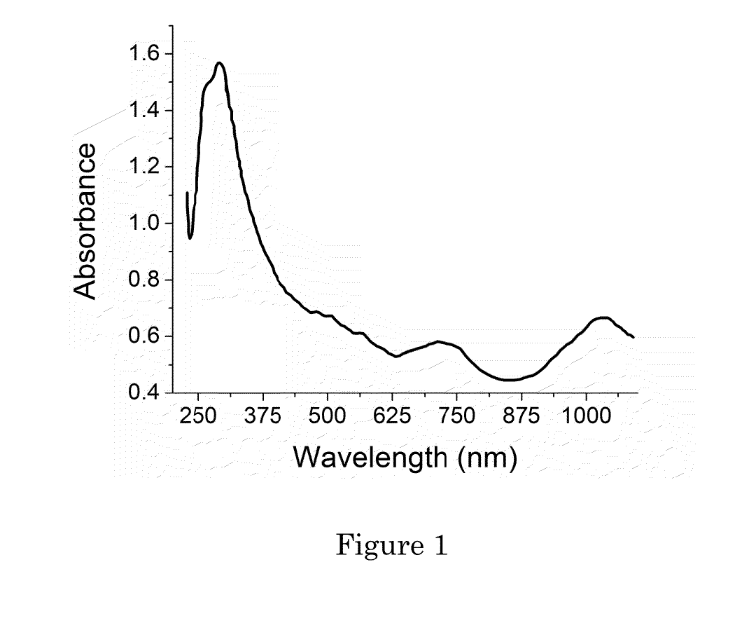

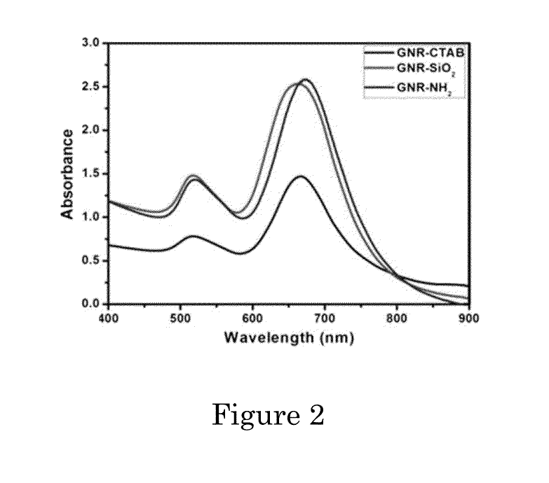

### DETAILED DESCRIPTION

Nanoparticle (NP) size 20 to 400 nm or 20 nm to 800 nm or 20 nm to 1500 nm. Sizes are diameters measured by dynamic light scattering and are the maximum average size for the distribution reported as the intensity weighted distribution. Examples of instruments for these measurements are the Malvern Nanosizer (Malvern Instruments Inc., Danvers Mass.), but other dynamic light scattering instruments are also suitable for measurements.

Hydrophobic core into which the hydrophobic photoacoustic imaging (PAI) agent is encapsulated.

Agents are incorporated into nanoparticles at mass concentrations of 0.001 wt %, or 0.01 wt %, or 0.1 wt %, or 1 wt %, or 10 wt %, or 50 wt %, or 99 wt % based on the total mass of the nanoparticle.

Hydrophobic is defined as having a solubility in DI water of less than 0.1 wt %, or less than 0.05 wt %, or less than 0.01 wt % in DI water at 20° C.

A photoacoustic imaging agent is an agent with the required properties to be excited by light in the wavelength range of 700 nm to 1100 nm and which is useful for imaging by photo excitation and acoustic detection. The agents are described in more details in section i. They are organic compounds or organic compounds with inorganic atoms coordinated into their structure. They are not predominantly inorganic metals or metal oxides or silicon or silicon-oxide materials.

NP stabilized by amphiphilic polymers. May be block, or comb graft (adapted from Mayer, et al. (2013))[16]:

An “amphiphilic stabilizer” is a compound having a molecular weight greater than about 500 that has a hydrophilic region and a hydrophobic region. Preferably the molecular weight is greater than about 1,000, or greater than about 1,500, or greater than about 2,000. Higher molecular weight moieties, e.g., 25,000 g/mole or 50,000 g/mole, may be used. “Hydrophobic” is defined as above. “Hydrophilic” in the context of the present invention refers to moieties that have a solubility in aqueous solution of at least 1.0 mg/ml. Typical amphiphilic stabilizers are copolymers of hydrophilic regions and hydrophobic regions. Thus, in the amphiphilic stabilizer, the hydrophobic region, if taken alone, would exhibit a solubility in aqueous medium of less than 0.05 mg/ml and the hydrophilic region, if taken alone, would exhibit a solubility in aqueous medium of more than 1 mg/ml. Examples include copolymers of polyethylene glycol and polycaprolactone.

Typically, the stabilizer is a copolymer of a hydrophilic block coupled with a hydrophobic block. Nanoparticles formed by the process of this invention can be formed with graft, block or random amphiphilic copolymers. These copolymers can have a molecular weight between 1,000 g/mole and 50,000 g/mole or more, or between about 3,000 g/mole to about 25,000 g/mole, or at least 2,000 g/mole. Alternatively, the amphiphilic copolymers used in this invention exhibit a water surface tension of at least 50 dynes/cm2 at a concentration of 0.1 wt %.

Examples of suitable hydrophobic blocks in an amphiphilic copolymer include but are not limited to the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, poly(D,L lactide), poly (D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery, Wright, Bristol, 1987; Arshady, J. Controlled Release (1991) 17:1-22; Pitt, Int. J. Phar. (1990) 59:173-196; Holland, et al., J. Controlled Release (1986) 4:155-180); hydrophobic peptide-based polymers and copolymers based on poly(L-amino acids) (Lavasanifar, A., et al., Advanced Drug Delivery Reviews (2002) 54:169-190), poly(ethylene-vinyl acetate) (“EVA”) copolymers, silicone rubber, polyethylene, polypropylene, polydienes (polybutadiene, polyisoprene and hydrogenated forms of these polymers), maleic anhydride copolymers of vinyl methylether and other vinyl ethers, polyamides (nylon 6,6), polyurethane, poly(ester urethanes), poly(ether urethanes), poly(ester-urea). Particularly preferred polymeric blocks include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) For non-biologically related applications particularly preferred polymeric blocks include polystyrene, polyacrylates, and butadienes.

Examples of suitable hydrophilic blocks in an amphiphilic copolymer include but are not limited to the following: carboxylic acids including acrylic acid, methacrylic acid, itaconic acid, and maleic acid; polyoxyethylenes or poly ethylene oxide; polyacrylamides and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, polyvinyl pyrrolidone, starches and starch derivatives, dextran and dextran derivatives; polypeptides, such as polylysines, polyarginines, polyglutamic acids; poly hyaluronic acids, alginic acids, polylactides, polyethyleneimines, polyionenes, polyacrylic acids, and polyiminocarboxylates, gelatin, and unsaturated ethylenic mono or dicarboxylic acids.

Preferably the blocks are either diblock or triblock repeats. Preferably, block copolymers for this invention include blocks of polystyrene, polyethylene, polybutyl acrylate, polybutyl methacrylate, polylactic acid, copolymers of polylactic-polyglycolic acid, polycaprolactone, polyacrylic acid, polyoxyethylene and polyacrylamide. A listing of suitable hydrophilic polymers can be found in Handbook of Water-Soluble Gums and Resins, R. Davidson, McGraw-Hill (1980).

In graft copolymers, the length of a grafted moiety can vary. Preferably, the grafted segments are alkyl chains of 12 to 32 carbons or equivalent to 6 to 16 ethylene units in length. In addition, the grafting of the polymer backbone can be useful to enhance solvation or nanoparticle stabilization properties. A grafted butyl group on the hydrophobic backbone of a diblock copolymer of a polyethylene and polyethylene glycol should increases the solubility of the polyethylene block. Suitable chemical moieties grafted to the block unit of the copolymer comprise alkyl chains containing species such as amides, imides, phenyl, carboxy, aldehyde or alcohol groups. One example of a commercially available stabilizer is the Hypermer family marketed by Uniqema Co. The amphiphilic stabilizer could also be of the gelatin family such as the gelatins derived from animal or fish collagen.

a. May contain therapeutic agent or may contain hydrophobic co-excipient. Adapted from Mayer, et al. (2013):[16]

In one application of the constructs of the invention, the constructs are used to deliver non-pharmaceutical or non-diagnostic agents including but not limited to pigments, inks, pesticides, herbicides, probes (including fluorescent probes), ingredients for sunscreens, fragrances and flavor compounds.

In another application of the constructs of the invention, the constructs are used to deliver pharmaceuticals or diagnostics in vivo. In these cases, the active is a therapeutic agent or a diagnostic agent.

A wide variety of therapeutic agents can be included. These may be anti-neoplastic agents, anthelmintics agents, antibiotics, anticoagulants, antidepressants, antidiabetic agents, antiepileptics, antihistamines, antihypertensive agents, antimuscarinic agents, antimycobacterial agents, immunosuppressants, antithyroid agents, antiviral agents, anxiolytic sedatives, astringents, beta-adrenoceptor blocking agents, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, biphosphonates, protease inhibitors, prostaglandins, radio-pharmaceuticals, sex hormones, steroids, anti-allergic agents, stimulants, sympathomimetics, thyroid agents, vasodilators and xanthines, disulfide compounds, antibacterials, antivirals, nonsteroidal anti-inflammatory drugs, analgesics, anticoagulants, anticonvulsants, antiemetics, antifungals, antihypertensives, anti-inflammatory agents, antiprotozoals, antipsychotics, cardioprotective agents, cytoprotective agents, antiarrhythmics, hormones, immunostimulating agents, lipid-lowering agents, platelet aggregation inhibitors, agents for treating prostatic hyperplasia, agents for treatment of rheumatic disease, or vascular agents (Compendium of Pharmaceuticals and Specialties (35th Ed.) incorporated herein by reference).

“Anti-neoplastic agent” refers to moieties having an effect on the growth, proliferation, invasiveness or survival of neoplastic cells or tumors. Anti-neoplastic therapeutic agents often include disulfide compounds, alkylating agents, antimetabolites, cytotoxic antibiotics, drug resistance modulators and various plant alkaloids and their derivatives. Other anti-neoplastic agents are contemplated.

Anti-neoplastic agents include paclitaxel, an etoposide-compound, a camptothecin-compound, idarubicin, carboplatin, oxaliplatin, adriamycin, mitomycin, ansamitocin, bleomycin, cytosine arabinoside, arabinosyl adenine, mercaptopolylysine, vincristine, busulfan, chlorambucil, melphalan, mercaptopurine, mitotane, procarbazine hydrochloride, dactinomycin, mitomycin, plicamycin, aminoglutethimide, estramustine phosphate sodium, flutamide, leuprolide acetate, megestrol acetate, tamoxifen citrate, testolactone, trilostane, amsacrine, asparaginase, interferon, teniposide, vinblastine sulfate, vincristine sulfate, bleomycin, methotrexate, valrubicin, carzelesin, paclitaxel, taxotane, camptothecin, doxorubicin, daunomycin, cisplatin, 5-fluorouracil, methotrexate; anti-inflammatory agents such as indomethacin, ibuprofen, ketoprofen, flubiprofen, dichlofenac, piroxicam, tenoxicam, naproxen, aspirin, and acetaminophen; sex hormones such as testosterone, estrogen, progestone, estradiol; antihypertensive agents such as captopril, ramipril, terazosin, minoxidil, and parazosin; antiemetics such as ondansetron and granisetron; antibiotics such as metronidazole, and fusidic acid; cyclosporine; prostaglandins; biphenyl dimethyl dicarboxylic acid, carboplatin; antifungal agents such as itraconazole, ketoconazole, and amphotericin; steroids such as triamcinolone acetonide, hydrocortisone, dexamethasone, prednisolone, and betamethasone; cyclosporine, and functionally equivalent analogues, derivatives or combinations thereof.

Diagnostic agents may also be included as actives. These may comprise, for example, chelated metal ions for MRI imaging, radionuclides, such as 99Tc or 111In or other biocompatible radionuclides. These may also be therapeutic agents.

“Hydrophobic moiety” is defined as a moiety which is insoluble in aqueous solution as defined above. The hydrophobic moiety may be a hydrophobic polymer such as polycaprolactone or may be a hydrophobic small molecule such as a vitamin or a steroid. It may be monovalent—i.e., have a suitable functional group for coupling only to a single active through a linker- or may be multivalent—i.e., able to couple to multiple actives through a linker. Not all of the actives need be the same.

The hydrophobic moiety may include polymers or natural products. Examples of suitable hydrophobic polymeric moieties include but are not limited to polymers of the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, and the polymers poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery, Wright, Bristol, 1987; Arshady, J. Controlled Release (1991) 17:1-22; Pitt, Int. J. Phar. (1990) 59:173-196; Holland, et al., J. Controlled Release (1986) 4:155-180); hydrophobic peptide-based polymers and copolymers based on poly(L-amino acids) (Lavasanifar, A., et al., Advanced Drug Delivery Reviews (2002) 54:169-190), poly(ethylene-vinyl acetate) (“EVA”) copolymers, silicone rubber, polyethylene, polypropylene, polydienes (polybutadiene, polyisoprene and hydrogenated forms of these polymers), maleic anhydride copolymers of vinyl-methylether and other vinyl ethers, polyamides (nylon 6,6), polyurethane, poly(ester urethanes), poly(ether urethanes), poly(ester-urea). Particularly preferred polymeric hydrophobes include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) For non-biologically related applications particularly preferred polymeric carriers include polystyrene, polyacrylates, and butadienes. The polymers must contain one or more functionizable groups which may be incorporated into the polymer by derivitization or may be inherent in the polymer chemistry. Polymers as hydrophobic moieties should have molecular weights between 800 and 200,000. The preferred range is 1,000 to 10,000 for polymers with mono or divalent functional sites. For polymers with a multiplicity of functional sites for derivation the preferred molecular weight of the polymer per conjugated active is 1,000 to 10,000.

Natural products with functional groups or groups that can be converted to functional groups for conjugation include: hydrophobic vitamins (for example vitamin E, vitamins K and A), carotenoids and retinols (for example beta carotene, astaxanthin, trans and cis retinal, retinoic acid, folic acid, dihydrofolate, retinyl acetate, retinyl palmitate), cholecalciferol, calcitriol, hydroxycholecalciferol, ergocalciferol, α-tocopherol, α-tocopherol acetate, α-tocopherol nicotinate, and estradiol. The preferred natural product is vitamin E which can be readily obtained as a vitamin E succinate, which facilitates functionalization to amines and hydroxyls on the active species.

Hydrophobic, non-polymeric and moieties include hydrocarbon molecules with solubilities less than 0.1 mg/ml that contain a functional group for can be derivatized to incorporate a functional group for conjugation. Molecules in this class include hydrophobic PAI agents and plasticizers. Examples include, but are not limited to, coumarin, diaminonaphthalene and other naphthalene derivatives, anthracene and its derivatives, nile red. Further examples can be found in The Sigma-Aldrich handbook of stains, dyes, and indicators.[17] Examples of hydrophobic plasticizers include dioctylphthalate, dibutylphthalate, and its derivatives.

Depending on the nature of the hydrophobic moiety, it may be able to accommodate more than one, including substantially more than one active through a multiplicity of linking sites. Polymeric moieties may have as many as 100 sites whereby actives could be linked. Simpler hydrophobic moieties, such as Vitamin E, may provide only one such site. Thus, the number of actives coupled to a single hydrophobic moiety may be only 1, or may be 2, 5, 10, 25, 100 and more, and all integers in between. For instance, the polymers set forth above can readily be provided with a multiplicity of functional groups for coupling to the active. Difunctional hydrophobic moieties would include the hydrophobic polymer chains listed above that have two terminal OH, COOH, or NH2 groups. Multifunctional hydrophobic moieties include all of those listed above that have multiple OH, COOH, or NH2 groups on some or all of the monomer units on the polymer backbone. These functional groups are merely illustrative; other moieties which could form functional groups for linking include phenyl substituents, halo groups, and the like. Typically, when the hydrophobic moiety is a hydrophobic polymer, it may have multiple sites for linkage. When the hydrophobic moiety is a relatively small molecule, it will accommodate only the number of linkers for which it has available functional groups.

b. May contain additional imaging agents well known in the fields of MRI (magnetic resonance imaging), PET (positron emission tomography) imaging, SPECT (single photon emission computed tomography) imaging, and X-ray imaging. These include but are not limited to:

MRI: Superparamagnetic iron oxide (SPIO), gadolinium, manganese

PET and SPECT: radioactive iodine, copper, zirconium, indium, yttrium, technetium, rhenium, gallium

X-ray: metals and metal oxides including but not limited to: gold, palladium, iron, cobalt ferrite

c. Amphiphilic polymer may have specific reactive sites on it for attaching targeting ligands. Or targeting ligands may be pre-attached to the amphiphilic polymer.

Attaching chemistry is well known in the field of targeting, comprising a wide variety of reactive groups such as, but not limited to, para-nitrophenol, maleimide, carboxylic acid, amine, azide, alkyne, mesylate and tosylate.

A wide variety of targeting molecules and constructs may be used. Non-limiting examples of these are small molecules, antibodies, single chain antibody fragments, peptides, nucleic acids, polymers/biopolymers, hormones and organometallic compounds.

d. NPs may be made by rapid precipitation (Adapted from Prud'homme, et al.)[18]

Flash NanoPrecipitation is a micromixing process comprising the steps of dissolving a hydrophobic organic compound in a compatible solvent, providing a polymer also dissolved in the solvent or in an aqueous solvent that is an anti-solvent to the organic compound, and rapidly micromixing the organic solution with the anti-solvent. The materials dissolved in the solvent(s), upon mixing in the anti-solvent, supersaturate the mixture and shortly precipitate into a population of uniformly sized nanoparticles. The kinetics of the process afford sufficient control to allow the artisan to mix hydrophobic organic compounds with amphiphilic polymers to produce nanoparticles of predictable size and stability (Johnson et al., Australian Journal of Chemistry 2003, 56: 1021-1024). The process has been disclosed and described in U.S. Patent Application Publication No. 2004/0091546 and in International Publication No.: WO 2006/014626, both of which are incorporated herein in their entirety by reference for all purposes.

In some embodiments, the present invention provides a process comprising the steps of dissolving a hydrophobic organic compound in a solvent, dispersing solid inorganic nanoparticles as a colloidal dispersion in that or another solvent, providing a polymer dissolved in that or another solvent (which may be an aqueous solvent that is an anti-solvent to the organic compound), and micromixing the organic solution, the dispersion and the anti-solvent such that polymeric nanoparticles are formed that retain, sterically stabilized therein, the hydrophobic organic compounds and the solid inorganic nanoparticles. Organics, including but not limited to organic fluorescent materials and therapeutic agents such as vitamins, anti-cancer agents, anti-bacterial agents, steroids, or analgesics may be incorporated into the composite nanoparticle. Solid inorganic nanoparticles including but not limited to imaging agents such as iron oxide nanoparticles, gold nanoparticles, gadolinium, and quantum dots may also be incorporated. Because the encapsulating nanoparticles of these embodiments are produced by means of flash nanoprecipitation, the use of surfactants to stabilize the nanoparticulate dispersions therein is unnecessary, uniformity of particle size is intrinsic to the process rather than a consequence of post-process purification, and the loading capacity for hydrophobic components is high.

The process of preparing multicomponent composite nano-particles may involve using a multi-inlet or multi-stream vortex mixer (described in Liu, et al. (2008) “Mixing in a multi inlet vortex mixer (MIVM) for Flash NanoPrecipitation”).[19] Alternatively, a confined impinging jet mixer as described in U.S. Patent Application Publication 2004/0091546 (incorporated herein in its entirety by reference for all purposes) can be employed.

Especially where unequal momentums of the organic and aqueous streams are advantageous, the multi-stream vortex mixer may be more suitable. Utilization of the multi-stream vortex mixer yields added flexibility in solvent selection, loading of multiple active agents and reduction of solvent to anti-solvent ratios. If two (or more) active agents are incompatible together in an otherwise convenient solvent, the two agents can be mixed from two separate solvent streams, and the velocity of each stream can be separately controlled. A constant flow rate can be provided by a syringe pump for each inlet tube using a Harvard Apparatus pump (model number 7023).

An exemplary but non-limiting multi-inlet vortex mixer, made of any rigid material, comprises a generally cylindrical mixing chamber 0.2333 inches in diameter and 0.0571 inches in height. The chamber is defined by a surrounding wall, a first cover or plate sealably disposed in orthogonal relation to the wall and, opposed thereto, a second sealable cover or plate. Four hollow cylindrical inlet tubes, each 0.0443 inches in diameter, penetrate the wall of the mixing chamber tangentially and, preferably, equidistantly, and are in fluid communication with the chamber. A hollow cylindrical outlet tube, 0.052 inches in diameter, has its long axis (approximately 0.5 inches in length) disposed in orthogonal relation to the inlet tubes. The outlet tube sealably penetrates one of the plates centrally and is in fluid communication with the chamber.

In some embodiments, a confined impinging jet mixer is suitable. A constant flow rate is provided by a syringe pump for each inlet tube using a Harvard Apparatus pump (model number 7023). At least one 100 ml glass syringe (SGE Inc.) is connected to each inlet tube. Two solvent streams of fluid are introduced into a mixing vessel through independent inlet tubes having a diameter, d, which can be between about 0.25 mm to about 6 mm but are between about 0.5 mm to about 1.5 mm in diameter for laboratory scale production. The solvent streams are impacted upon each other while being fed at a constant rate from the inlet tube into the mixing vessel. The mixing vessel is a cylindrical chamber with a hemispherical top. The diameter of the mixing vessel, D, is typically between 2.0 mm to about 5.0 mm, but preferably is between about 2.4 mm to about 4.8 mm. The mixing vessel also contains an outlet with a diameter, ?, that can be between about 0.5 mm to about 2.5 mm but is preferably between 1.0 mm to about 2.0 mm. The outlet of the mixer is connected to an 8-inch line of ⅛th-inch (tubing leading out for product collection.

The organic solutes, inorganic nanostructures and amphiphilic copolymers are dissolved, solubilized or dispersed, together or separately, in a water-miscible organic solvent including but not limited to tetrahydrofuran, dimethyl sulfoxide, or ethanol. Other pharmaceutically acceptable water-miscible solvents are listed in U.S. Pat. No. 6,017,948, which is incorporated herein in its entirety by reference for all purposes. In preferred embodiments, the inorganic nanostructures (generally sized between 1 nm to 700 nm) are “pre-formed” or “pre-existing” in the sense that they retain their discrete particulate nature when solubilized (dispersed) in the water-miscible organic phase and continue to retain it after being incorporated into the nanoparticulate product, even if that product simultaneously encapsulates organics. Pre-formed nanostructures are not formed during the nanoprecipitation process but beforehand.

Intense mixing (i.e., the mixing system operates at a Reynolds number>1600 where the Reynolds number is appropriately defined) of the organic solvent stream with water or a predominantly aqueous stream in the multi-inlet vortex mixer induces, in milliseconds, highly supersaturated mixtures (a solute “supersaturates” a solvent when the ratio of the concentration of the solute initially in the mixed streams in the mixing chamber to the concentration of the solute at equilibrium in the final solvent mixture is greater than 1). The artisan can readily measure the stream velocities of the inlet streams and the kinematic viscosity of each stream by means well known in the art, and can determine therefrom the Reynolds number for the system, defined as the sum of the stream flow rates times the average density of the fluids therein divided by the diameter of the inlet stream and divided by the average fluid viscosity of the streams. When solutes mixed under these conditions precipitate from a supersaturated state, nanoparticles of uniform size emerge. They are stable and remain dispersed as they leave the outlet tube. Actives captured within the particles also remain stable.

It is well within the skill of the artisan to “tune” the described mixing system to cause it to produce nanoparticles of a size between 1 nm and 10,000 nm (but preferably less than 1000 nm). A method (“dynamic light scattering”) for determining sizes of nanoparticles in the context of the relevant embodiments of the invention is well known in the art. Thus, the artisan can select a size distribution that covers a fraction of this spectrum by tuning the system through solvent selection, choice of solute concentrations, stream velocities, conditions of temperature and pressure, and “time-scaling” as described in detail below.

The stability of the nanoparticles is also within the artisan's control, principally through the selection of polymers. In preferred embodiments, amphiphilic polymers or polymer systems are used. The relative sizes (molecular weights) of their hydrophilic and hydrophobic domains determines stability. The particles tend toward instability as hydrophobic domains are made smaller. As hydrophilic domains are made smaller, the particles may remain stable internally but, in dispersions, they will tend to aggregate and flocculate.

e. NPs may be made by emulsion stripping process (Adapted from Troiano, et al.)[20]

In another embodiment, a nanoemulsion process is provided. For example, a therapeutic agent, a first polymer (for example, a diblock co-polymer such as PLA-PEG or PLGA-PEG, either of which may be optionally bound to a ligand) and an optional second polymer (e.g. (PL(G)A-PEG or PLA), with an organic solution to form a first organic phase. Such first phase may include about 5 to about 50% weight solids, e.g., about 5 to about 40% solids, or about 10 to about 30% solids. The first organic phase may be combined with a first aqueous solution to form a second phase. The organic solution can include, for example, toluene, methyl ethyl ketone, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80, or the like, and combinations thereof. In an embodiment, the organic phase may include benzyl alcohol, ethyl acetate, and combinations thereof. The second phase can be between about 1 and 50 weight %, e.g., about 5-40 weight %, solids. The aqueous solution can be water, optionally in combination with one or more of sodium cholate, ethyl acetate, polyvinyl acetate and benzyl alcohol.

For example, the oil or organic phase may use solvent that is only partially miscible with the nonsolvent (water). Therefore, when mixed at a low enough ratio and/or when using water pre-saturated with the organic solvents, the oil phase remains liquid. The oil phase may be emulsified into an aqueous solution and, as liquid droplets, sheared into nanoparticles using, for example, high energy dispersion systems, such as homogenizers or sonicators. The aqueous portion of the emulsion, otherwise known as the “water phase”, may be surfactant solution consisting of sodium cholate and pre-saturated with ethyl acetate and benzyl alcohol.

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizer, e.g. by using 1, 2, 3 or more passes through a homogenizer. For example, when a high pressure homogenizer is used, the pressure used may be about 1000 to about 8000 psi, about 2000 to about 4000 psi 4000 to about 8000 psi, or about 4000 to about 5000 psi, e.g., about 2000, 2500, 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5 degrees Celsius or less. For example, water used in the quenching may be at a temperature that is less that room temperature (e.g. about 0 to about 10 degrees Celsius, or about 0 to about 5 degrees Celsius).

In some embodiments, not all of the therapeutic agent (e.g. docetaxel) is encapsulated in the particles at this stage, and a drug solubilizer is added to the quenched phase to form a solubilized phase. The drug solubilizer may be for example, Tween 80, Tween 20, polyvinyl pyrrolidone, cyclodextran, sodium dodecyl sulfate, or sodium cholate. For example, Tween-80 may be added to the quenched nanoparticle suspension to solubilize the free drug and prevent the formation of drug crystals. In some embodiments, a ratio of drug solubilizer to therapeutic agent (e.g. docetaxel) is about 100:1 to about 10:1.

The solubilized phase may be filtered to recover the nanoparticles. For example, ultrafiltration membranes may be used to concentrate the nanoparticle suspension and substantially eliminate organic solvent, free drug, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (about 5-25 nm) may be used.

f. The unique characteristic of PAI agents we claim are those with an absorption band in the wavelength range 700-1100 nm, but which also have an absorption that is 15% or less of the maximum absorption band in the wavelength range 700-1,100 nm. The definition of 15% or less absorbance is determined from an absorbance measurement done at a nanoparticle or dye concentration such that the absorbance maxima in the wavelength range is AU=1 or higher. At this concentration, a minimum in absorbance in the wavelength range 700-1100 nm will be 15% or less above the background absorbance in the absence of dye or nanoparticle. This strong adsorption plus minimal adsorption in this wavelength region enables good background signal acquisition using a laser wavelength where the PAI agent only weakly absorbs, and then more precise location of the targeted nanoparticles by tuning the laser exciation to the region of stronger PAI agent adsorption. This enables higher resolution than can be obtained from PAI agents that have broad adsorption over the desired wavelength so that background subtraction is less efficient. Examples of dyes which may be used in the present invention are:

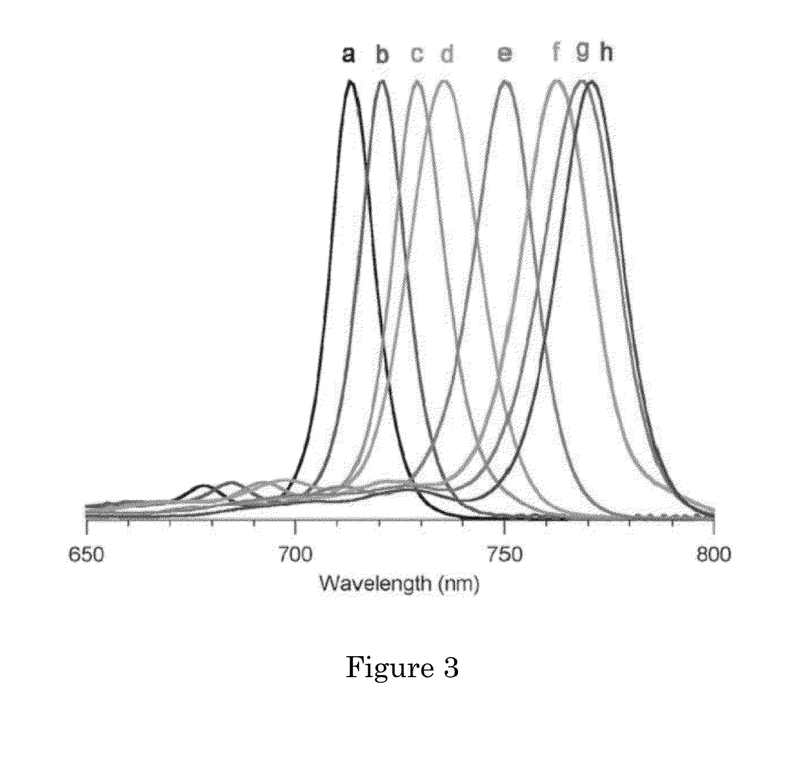

i. Bacteriochlorins: PAI agents molecules comprising the bacteriochlorin structure as described in Taniguchi, et al. (2008)[3]. Spectrum shown in FIG. 3.

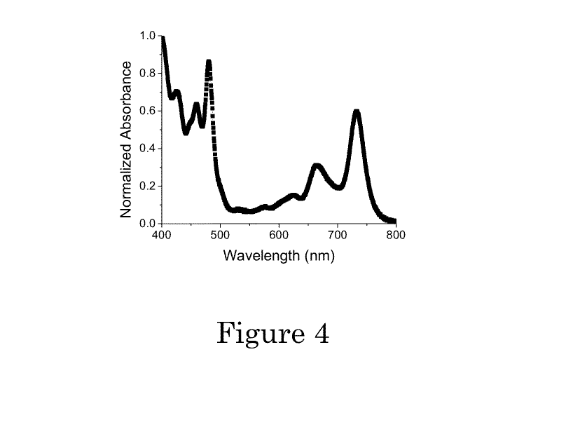

ii. John Anthony LW2: PAI agents molecules comprising hexacene-derived structures as described in Payne (2005)[21] and Wolak (2006)[22]. Spectrum shown in FIG. 4.

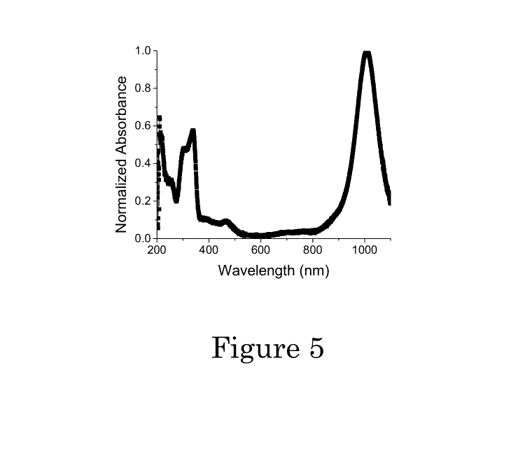

iii. IR122 PAI agent (BASF). Spectrum shown in FIG. 5.

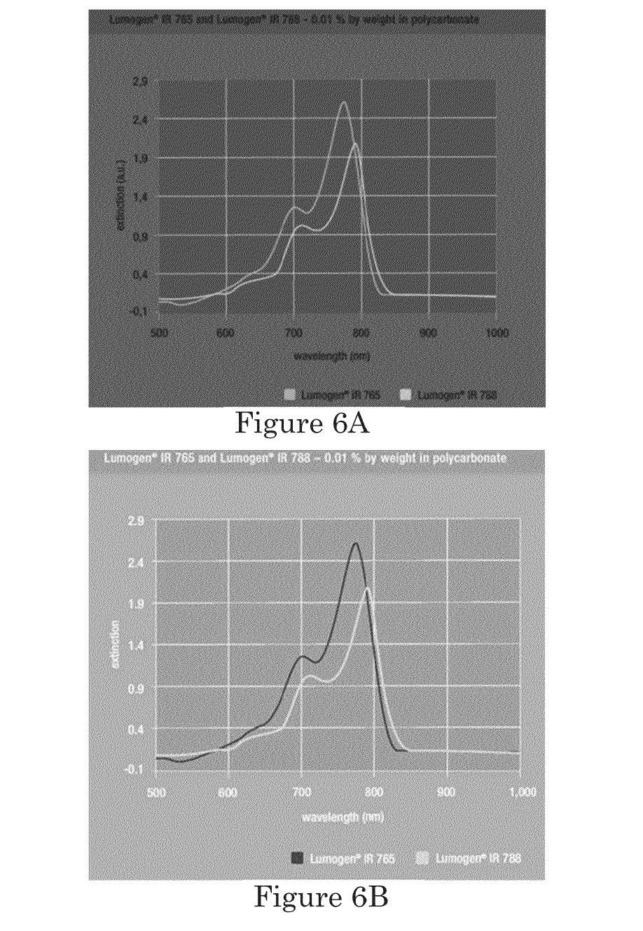

iv. BASF Lumogen series of PAI agents (including but not limited to Lumogen 765 and Lumogen 788). FIGS. 6A and 6B are graphs showing the unencapsulated absorbance spectra for BASF Lumogen 765 and 788 PAI agents.

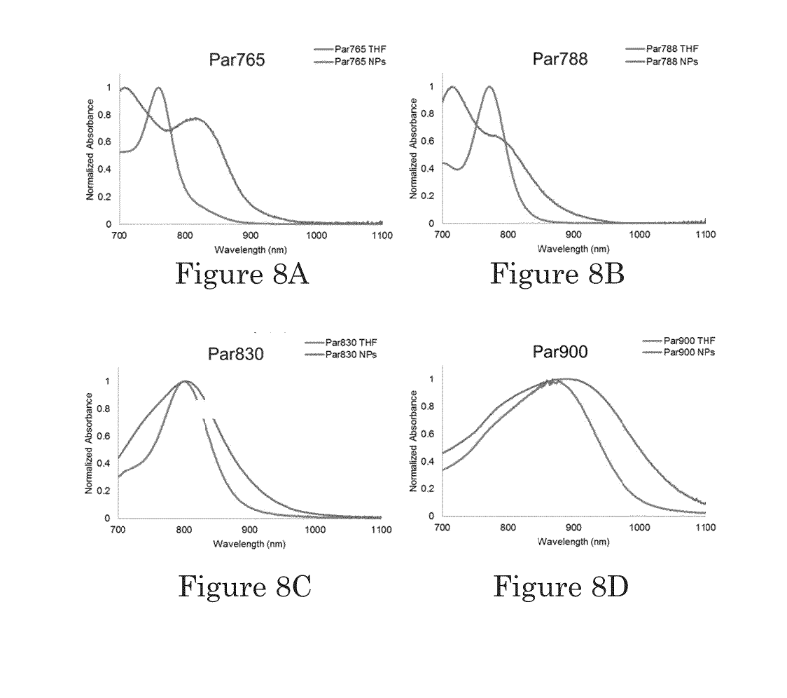

v. Persis Science Inc. Par series of PAI agents (including but not limited to Par765, Par788, Par830, Par900). FIGS. 8A-8D are graphs showing Normalized absorbance spectra of unencapsulated PAI dyes and encapsulated PAI dyes in nanoparticles using commercially available: FIG. 8A—Par765, FIG. 8B—Par788, FIG. 8C—Par830, and FIG. 8D—Par900 dyes from Persis Science Inc. Par900 NPs absorb at a maximum value at 890 nm wavelength, and absorb 9.8% the maximum value at 1100 nm wavelength.

g. The PAI agent is hydrophobic or may be made hydrophobic by ion pairing (as described in Pinkerton, et al.) or conjugation to a hydrophobic anchor

Hydrophobic Linkage (Adapted from Mayer, et al. (2005))[16]

A “linker” refers to any covalent bond, to a divalent residue of a molecule, or to a chelator (in the case where the active is a metal ion or organic metallic compound, e.g., cisplatin) that allows the hydrophobic moiety to be attached to the active agent. The linker may be selectively cleavable upon exposure to a predefined stimulus, thus releasing the active agent from the hydrophobic moiety. The site of cleavage, in the case of the divalent residue of a molecule may be at a site within the residue, or may occur at either of the bonds that couple the divalent residue to the agent or to the hydrophobic moiety. The predefined stimuli include, for example, pH changes, enzymatic degradation, chemical modification or light exposure. Convenient conjugates are often based on hydrolyzable or enzymatically cleavable bonds such as esters, carbonates, carbamates, disulfides and hydrazones.

In some instances, the conditions under which the active performs its function are not such that the linker is cleaved, but the active is able to perform this function while still attached to the particle. In this case, the linker is described as “non-cleavable,” although virtually any linker could be cleaved under some conditions; therefore, “non-cleavable” refers to those linkers that do not necessarily need to release the active from the particle as the active performs its function.

The linker component, as described above, may be or may include a cleavable bond.

The linker may be, for example, cleaved by hydrolysis, reduction reactions, oxidative reactions, pH shifts, photolysis, or combinations thereof; or by an enzyme reaction. Some linkers can be cleaved by an intracellular or extracellular enzyme, or an enzyme resulting from a microbial infection, a skin surface enzyme, or an enzyme secreted by a cell, by an enzyme secreted by a cancer cell, by an enzyme located on the surface of a cancer cell, by an enzyme secreted by a cell associated with a chronic inflammatory disease, by an enzyme secreted by a cell associated with rheumatoid arthritis, by an enzyme secreted by a cell associated with osteoarthritis, or by a membrane-bound enzyme. In some cases, the linker can be cleaved by an enzyme that is available in a target region. These types of linkers are often useful in that the particular enzyme or class of enzymes may be present in increased concentrations at a target region. The target tissue generally varies based on the type of disease or disorder present in the subject.

The linker may also comprise a bond that is cleavable under oxidative or reducing conditions, or may be sensitive to acids. Acid cleavable linkers can be found in U.S. Pat. Nos. 4,569,789 and 4,631,190; and Blattner, et al., Biochemistry (1984) 24:1517-1524. Such linkers are cleaved by natural acidic conditions, or alternatively, acid conditions can be induced at a target site as explained in U.S. Pat. No. 4,171,563.

A non-limiting set of molecules that can form acid cleavable bonds include cis-polycarboxylic alkenes (see U.S. Pat. No. 4,631,190), and amino-sulfhydryl cross-linking reagents which are cleavable under mildly acidic conditions (see U.S. Pat. No. 4,569,789). The linker may comprise a time-release bond, such as a biodegradable and/or hydrolyzable bond, such as esters, amides or urethane bonds.

Examples of linking reagents which contain cleavable disulfide bonds (reducible bonds) include 1,4-di-[3?-(2?-pyridyldithio)propionamido]butane; N-succinimidyl(4-azidophenyl) 1,3?-dithiopropionate; sulfosuccinimidyl (4-azidophenyldithio)propionate; dithiobis(succinimidylpropionate); 3,3?-dithiobis(sulfosuccinimidylpropionate); dimethyl 3,3?-dithiobispropionimidate-2HCl (available from Pierce Chemicals, Rockford, Ill.).

Examples of oxidation sensitive linking reagents include, without limitation, disuccinimidyl tartarate; and disuccinimidyl tartarate (available from Pierce Chemicals).

The linker may also comprise a small molecule such as a peptide linker. Frequently, in such embodiments, the peptide linker is cleavable by base, under reducing conditions, or by a specific enzyme. The linker may be cleaved by an indigenous enzyme, or by an non-indigenous enzyme administered after or in addition to the presently contemplated compositions. A small peptide linker is pH sensitive, for example, the linker may comprise linkers selected from the group consisting of poly L-glycine; poly L-glutamine; and poly L-lysine linkers.

For example, the linker may comprise a hydrophobic polymer and a dipeptide, L-alanyl-L-valine (Ala-Val), cleavable by the enzyme thermolysin. This linker is advantageous because thermolysin-like enzyme has been reported to be expressed at the site of many tumors. A linker may also be used that contains a recognition site for the protease furin. Goyal, et al., Biochem. J. (2000) 2:247-254.

The chemical and peptide linkers can be bonded between the ligand and the agent by techniques known in the art for conjugate synthesis, i.e., using genetic engineering or chemically.

Photocleavable linkers include, for example, 1-2-(nitrophenyl)-ethyl. A photocleavable linker often permits the activation and action of the active agent in a very specific area, for example at a particular part of the target tissue. Activation (light) energy can be localized through a variety of means including catheterization, via natural or surgical openings or via blood vessels.

The linkers and techniques for providing coupling of the active to the hydrophobic moiety are similar to those that have been used previously to prepare conjugates to make actives more soluble, in contrast to their application in the present invention. In general, in the constructs of the invention, the active is often, but not always, made less soluble in aqueous solution by virtue of forming the conjugate. For example, the techniques reviewed by Greenwald, et al., for attaching PEG to small organic molecules can be adapted to the present invention. Some of these techniques are described in Greenwald, R. B., Journal of Controlled Release (2001) 74:159-171; Greenwald, R. B., et al., Journal of Medicinal Chemistry (1996) 39:424-431; and Greenwald, R. B., et al., Advanced Drug Delivery Reviews (2002) 55:217-250. In particular, paclitaxel esters have been prepared via conjugation of PEG acids to the ?-position on the paclitaxel molecule. These esters were demonstrated to be an especially effective linking group, as hydrolysis of the ester carbonyl bond and the subsequent release of the attached drug were shown to occur in a predictable fashion in vitro. (Greenwald, R. B., et al., Critical Reviews in Therapeutic Drug Carrier Systems (2000) 17:101-161.) The linker chemistry as applied in the present invention does not enhance solubility, but adapts the active for inclusion in the particulate vehicles of the invention.

The covalent attachment of proteins, vaccines or peptides to PEG can also be adapted to form the present conjugate. Such techniques are reviewed in Katre, N. V., Advanced Drug Delivery Reviews (1993) 10:91-114; Roberts, M. J., et al., Journal of Pharmaceutical Sciences (1998) 87:1440-1445; Garman, A. J., et al., Febs Letters (1987) 223:361-365; and Daly, S. M., et al., Langmuir (2005) 21:1328-1337. Coupling reactions between amino groups of proteins and mPEG equipped with an electrophilic functional group have been used in most cases for preparation of PEG-protein conjugates. The most commonly used mPEG-based electrophiles, referred to as ‘activated PEGs’ are based on reactive aryl chlorides, acylating agents and alkylating groups as described by Zalipsky, S., Advanced Drug Delivery Reviews (1995) 16:157-182; and Zalipsky, S., Bioconjugate Chem. (1995) 6:150-165. Tailoring the number of ethylene groups in the linker can additionally be used to adjust the hydrolysis rates of drug-linked ester bonds, to values appropriate for once-a-week administration. For example, Schoenmakers, et al., demonstrated the conjugation of a model paclitaxel molecule to PEG using a hydrolysable linker based on reaction between a thiol and an acrylamide. By changing the length of the linker, the time of drug release was varied between 4 and 14 days. (Schoenmakers, R. G., et al., Journal of Controlled Release (2004) 95:291-300.) Additionally, Frerot, et al., prepared a series of carbamoyl esters of maleate and succinate and studied the rate constants for neighboring group assisted alkaline ester hydrolysis. The rates of hydrolysis were found to depend on the structure of the neighboring nucleophile that attacks the ester function. (de Saint Laumer, J. Y., et al., Helvetica Chimica Acta (2003) 86:2871-2899.) By taking account of the influence of structural parameters on the rates of ester hydrolysis, hydrolysis rates may be varied over several orders of magnitude and precursors yielding the desired release profile may be designed.

In addition to ester linkages, enzymatically cleavable bonds can be used to conjugate active agents to the hydrophobic moiety. An enzymatically cleavable linker generally will comprise amino acids, sugars, nucleic acids, or other compounds which have one or more chemical bonds that can be broken via enzymatic degradation. In a recent study, a variety of amino acid spacers were employed for the conjugation of PEG to camptothecin, an anti-tumor drug. Rates of amino acid linker hydrolysis were determined to vary according to the type of amino acid spacer utilized. (Conover, C. D., et al., Anti-Cancer Drug Design (1999) 14:499-506.)

Photocleavable linkers have also been extensively employed for the synthesis of conjugates for release of actives. As an example, keto-esters have been used as delivery systems for the controlled release of perfumery aldehydes and ketones. Alkyl or aryl ?-keto esters of primary or secondary alcohols decompose upon radiation at 350-370 nm, releasing the active aldehyde. (Rochat, S., et al., Helvetica Chimica Acta (2000) 83:1645-1671.) This mechanism has been shown to successfully sustain release of the active agent. For drug delivery purposes, light energy can be localized through a variety of means including catheterization, via natural and surgical openings or via blood vessels.

As noted above, when the linker is the residue of a divalent organic molecule, the cleavage “of the linker” may be either within the residue itself, or it may be at one of the bonds that couples the linker to the remainder of the conjugate—i.e., either to the active or the hydrophobic moiety.

In some embodiments, it is unnecessary for the linker to be cleavable. In particular, if the active is functional while still coupled to the linker, there is no need to release the active from the particulate moiety. One such example would be instances wherein the active is printer's ink, which can remain in particulate form when employed.

In instances where the linker need not be cleavable, alternative organic moieties may be used to create the divalent residue, or a covalent bond directly coupling the active to the hydrophobic moiety may not be subject to cleavage under conditions contemplated in use. (By “non-cleavable” is meant that the linker will not release the active under the conditions wherein the function of the active is being performed.) Examples of non-cleavable linkers comprise, but are not limited to, (sulfosuccinimidyl 6-[alpha-methyl-alpha-(2-pyridylthio)toluamido]hexanoate; Azidobenzoyl hydrazide; N-Hydroxysuccinimidyl-4-azidosalicyclic acid; Sulfosuccinimidyl 2-(p-azidosalicylamido)ethyl-1,3-dithiopropionate; N-{4-(p-azidosalicylamido) buthy}maxima-3?(2?-pyidyldithio)propionamide; Bis-[beta-(4-azidosalicylamido)ethyl]disulfide; N-hydroxysuccinimidyl-4 azidobenzoate; p-Azidophenyl glyoxal monohydrate; N-Succiminidyl-6(4?-azido-2?-mitrophenyl-amino)hexanoate; Sulfosuccinimidyl 6-(4?-azido-2?-nitrophenylamino)hexanoate; N-5-Azido-2-nitrobenzyoyloxysuccinimide; Sulfosuccinimidyl-2-(m-azido-o-mitrobenzamido)-ethyl-1,3?-dithiopropionate; p-nitrophenyl-2-diazo-3,3,3-trifluoropropionate; Succinimidyl 4-(N-maleimidomethyl)cyclohexane-1-carboxylate; Sulfosuccinimidyl 4-(N-maleimidomethyl)cyclohexane-1-carboxylate; m-Maleimidobenzoyl-N-hydroxysuccinimide ester; m-Maleimidobenzoyl-N-hydroxysulfosuccinimide ester; N-Succinimidyl(4-iodoacetyl)aminobenzoate; N-Sulfosuccinimidyl(4-iodoacetyl)aminobenzoate; Succinimidyl 4-(p-malenimidophenyl)butyrate; Sulfosuccinimidyl 4-(p-malenimidophenyl)butyrate; Disuccinimidyl suberate; bis(sulfosuccinimidyl) suberate; Bis maleimidohexane; 1,5-difluoro-2,4-dinitrobenzene; dimethyl adipimidate 2HCl; Dimethyl pimelimidate-2HCl; dimethyl suberimidate-2-HCl; “SPDP”-N-succinimidyl-3-(2-pyridylthio)propionate; Sulfosuccinimidyl 4-(p-azidophenyl)butyrate; Sulfosuccinimidyl 4-(p-azidophenylbutyrate); 1-9p-azidosalicylamido)-4-(iodoacetamido)butane; 4-(p-Azidosalicylamido)butylamine (available from Pierce Chemicals).

Ion Pairing

In cases where the active is not sufficiently hydrophobic to form uniform nanoparticles according to the methods outlined in sections (g—Flash NanoPrecipitation) and (h—Emulsion Stripping), the active can be formulated with a counter ionic species to form a hydrophobic salt in situ that will render the active-counter ion complex amenable to forming nanoparticles by Flash NanoPrecipitation or emulsion stripping. Non-limiting examples of counter ionic species are: (±)-camphor-10-sulfonic acid, pamoic acid, cinnamic acid, palmitic acid, oleic acid, and N,N′-dibenzyl-ethylenediamine.[23]

h. The constructs are useful for PAI.

i. Multiple PAI NPs can be used, each with a distinct PAI agent with absorbance maxima in the range 700-1400 nm, and each with a different targeting ligand. In a single experiment the concentration of each NP at sites of interest can be determined. This enables multiplexing and gives much more extensive information on tissue types.

### EXAMPLES

### Example 1

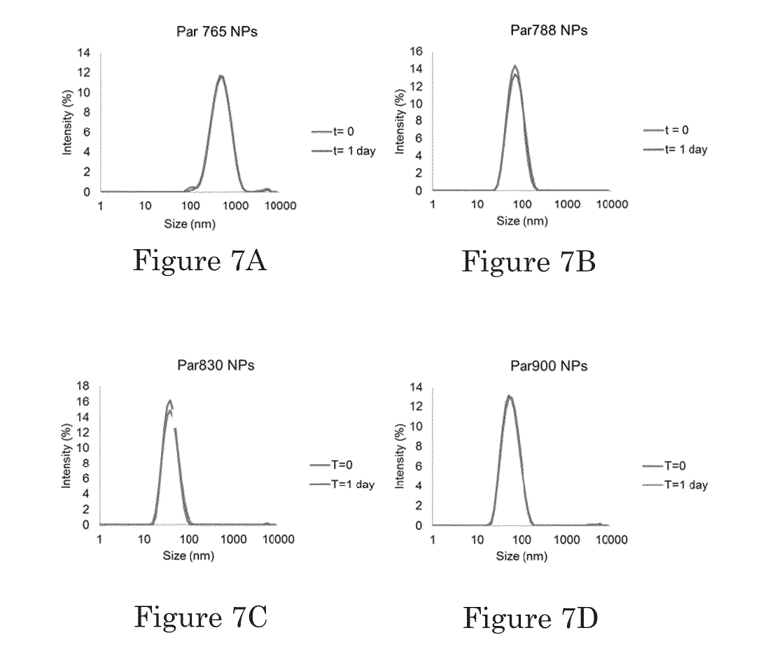

FIGS. 7A-7D are graphs showing dynamic light scattering size distribution spectra of PAI nanoparticles formed using commercially available: FIG. 7A—Par765, FIG. 7B—Par788, FIG. 7D—Par830, and FIG. 7D—Par900 dyes from Persis Science Inc. Par series dyes are encapsulated using a polymeric 1.6 kDa polystyrene-block 5 kDa polyethylene glycol (PS_{1.6k}-b-PEG₅ₖ) stabilizer to form Par series NPs. Nanoparticles were formed using Flash NanoPrecipitation. A variety of dyes can be encapsulated to form PAI polymeric nanoparticles with a variety of size distributions. Particles are stable and retain similar sizes over the time course of one day at ambient temperature and pressure.

### Example 2

FIGS. 8A-8D are graphs showing normalized absorbance spectra of PAI dyes and PAI particles using commercially available: FIG. 8A Par765, FIG. 8B—Par788, FIG. 8C—Par830, and FIG. 8D—Par900 dyes from Persis Science Inc. Absorbance spectra of unencapsulated Par series PAI is taken when dissolved in tetrahydrofuran (Par series THF). Absorbance spectra of nanoparticle Par series PAI (Par series NPs) is taken when suspended in DI water when PAI is encapsulated using a polymeric 1.6 kDa polystyrene-block 5 kDa polyethylene glycol (PS_{1.6k}-b-PEG₅ₖ) stabilizer. A variety of PAI active NPs with a diverse set of absorbances can be formed. All constructs have a maximum peak absorbance within the 700-1100 nm wavelength window. All constructs have a low absorbance value that is below 15% of baseline absorbance within the 700-1100 nm wavelength window. Par900 NPs absorb at a maximum value at 890 nm wavelength, and 9.8% the maximum value at 1100 nm wavelength.

### Example 3

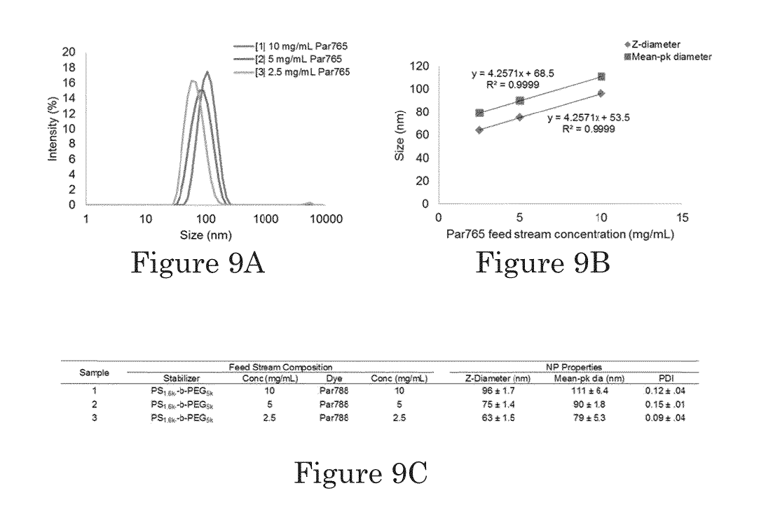

FIGS. 9A-9C are graphs showing how the nanoparticle size can be controlled by the rapid precipitation process Flash NanoPrecipitation including: FIG. 9A—Dynamic light scattering size distribution spectra and FIG. 9B—intensity-weighted diameters of PAI nanoparticles formed using Par765 PAI at different compositions. FIG. 9C is a table showing formulation summaries used to make Par765 NPs of varying sizes. Par765 was dissolved in THF at a 1:1 mass raio with PS_{1.6k}-b-PEG₅ₖ stabilizer and rapidly mixed with against water using a confined impingement jet. PAI NPs can be controllably made into varying sizes by tuning feed stream compositions during Flash NanoPrecipitation.

### Example 4

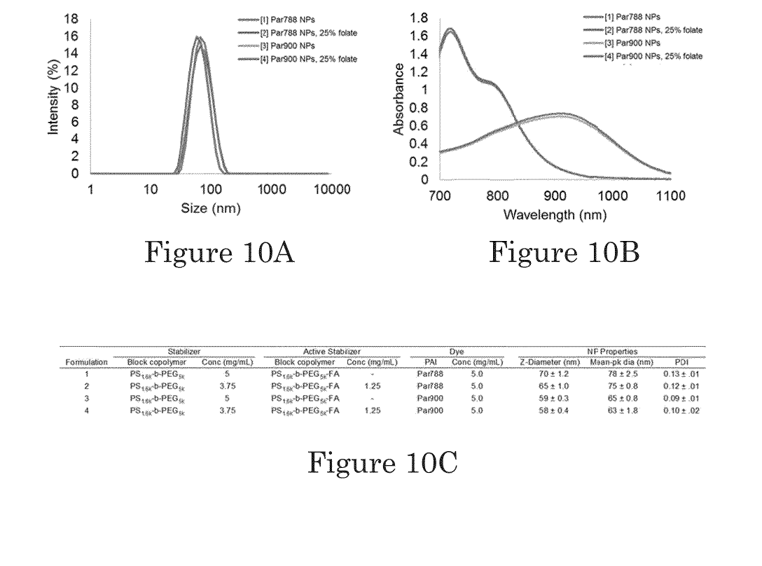

FIGS. 10A and 10B are graphs showing the formation of targeted PAI nanoparticles including: FIG. 10A—Dynamic light scattering size distribution spectra and FIG. 10B—absorbance spectra of Par series nanoparticles. FIG. 10C is table showing formulation summaries used to make targeting Par series NPs. Par788 or Par900 was dissolved in 50% DMSO 50% THF at a 1:1 mass raio with PS_{1.6k}-b-PEG₅ₖ stabilizer, or PS_{1.6k}-b-PEG₅ₖ stabilizer and 1.6 kDa polystyrene-block 5 kDa polyethylene glycol linked to folic acid (PS_{1.6k}-b-PEG₅ₖ-FA), and rapidly mixed with against water using a confined impingement jet. The addition of PS_{1.6k}-b-PEG₅ₖ-FA decorates the surface of the PAI NP with folic acid targeting moeities. PAI NPs can be formed with targeting functional moeties.

### Example 5

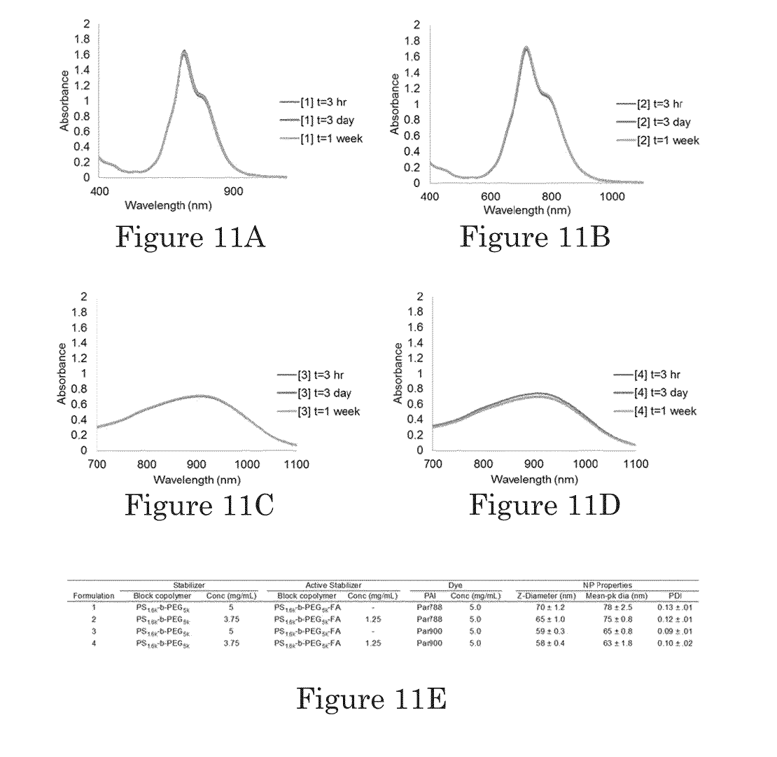

FIGS. 11A-11D are graphs showing the optical stability of targeting PAI NPs including the absorbance profiles of folic acid functionalized and non-functionalized control Par series NPs. E is table showing the formulation summaries used to make targeting Par series NPs of FIGS. 11A-11D. FIG. 11E is a table showing the formulation summaries used to make targeting Par series NPs. Particles are stable and retain similar absorbances over the time course of one up to one week at ambient temperature and pressure.

### Example 6

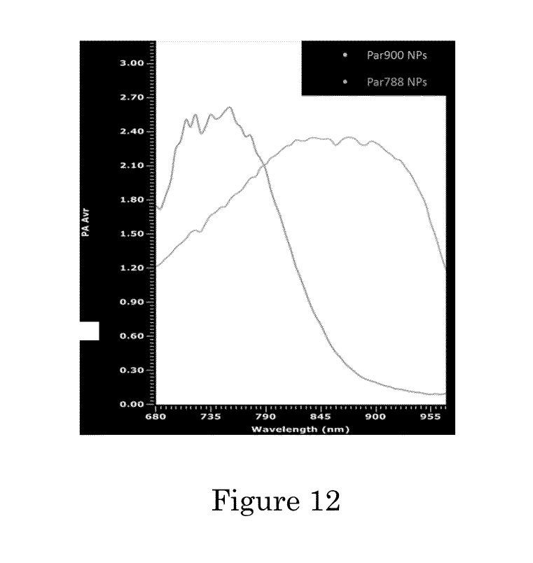

FIG. 12 is a graph showing the photoacoustic activity of Par series NPs. Photoacoustic activity of Par788 or Par900 NPs was measured using a Vevo LAZR Photoacoustic Imaging System. Particles exhibit photoacoustic activity.

### Example 7

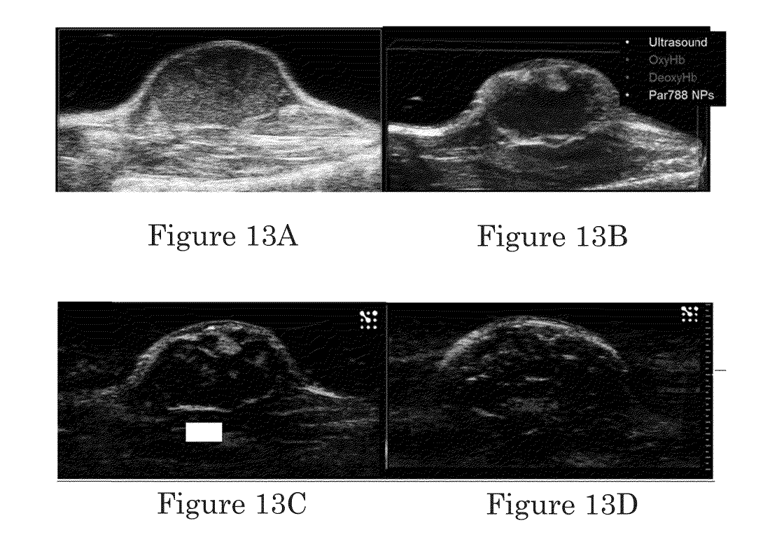

FIGS. 13A-13D are photos showing the imaging of tumors using photoacoustic NPs Including FIG. 13A—High resolution ultrasound-based visualization of the anatomy of a KB tumor implanted in a mouse; FIG. 13B—3D coregistered ultrasound/photoacoustic-based visualization of tumor anatomy & targeted nanoparticle distribution, after the mouse was injected with folate modified Par788 NPs; FIG. 13C—Spectrally unmixed photoacoustic image showing targeted nanoparticle distribution and blood signals. Panels (A-C) are of the same mouse; and FIG. 13D—Spectrally unmixed photoacoustic image showing non-targeted nanoparticle distribution and blood signals when non-targetd Par788 NPs were injected into a mouse implanted with a KB tumor. PAI NPs can be visualized in tumors using photoacoustic imaging. PAI imaging demonstrates PAI NPs preferentally accumulate in tumors. PAI NPs demonstrate photoacoustic activity. Images were taken with a Vevo LAZR Photoacoustic Imaging System. Greyscale=ultrasound, red=oxygentated hemoglobin, blue=deoxygenated hemoglobin, yellow=Par788 NPs (targeted or nontargeted).

### Example 8

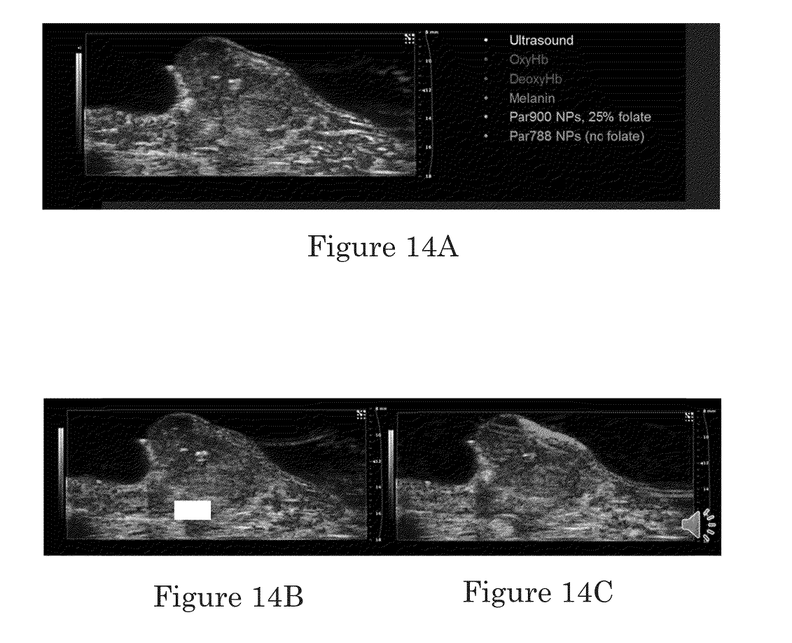

FIGS. 14A-14C are photos showing simultaneous imaging of a tumor using two photoacoustic NPs at different wavelengths including: FIG. 14A—Ultrasound image of a KB tumor implanted in a mouse with blood signals; FIG. 14B—Image of a KB tumor injected with folate targeting Par900 NPs, coregistered with Par900, ultrasound, and blood signals; and FIG. 14C—Image of a KB tumor injected with nontargeted Par788 NPs (after being injected with folate targeting Par900 NPs), coregistered with Par788, Par900, ultrasound, and blood signals. Multiple PAI NPs with different PAI activity and absorbance profiles can be imaged and co-registered in a single image. Images were taken with a Vevo LAZR Photoacoustic Imaging System. Greyscale=ultrasound, red=oxygentated hemoglobin, blue=deoxygenated hemoglobin, yellow=folate targeted Par900 NPs, green=nontargetted Par788 NPs. Note—color intensities between colors in images are not directly comparable to concentrations of each different component in mouse tumors.

### Example 9

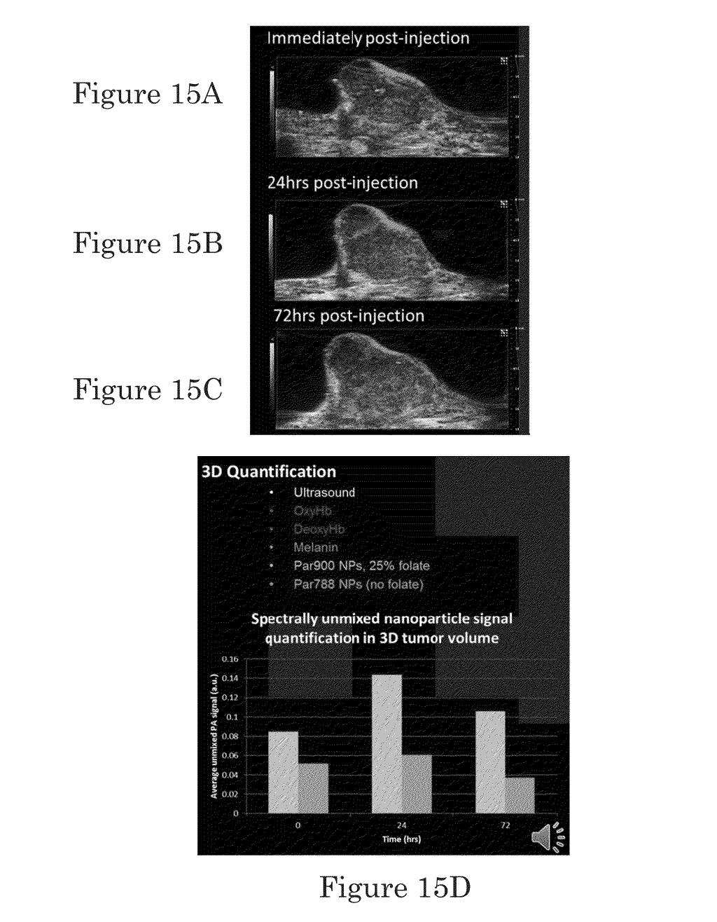

FIG. 15A-15C are photos showing the simultaneous imaging of a tumor using two photoacoustic NPs at different wavelengths over time. Image of a KB tumor implanted in a mice and injected with both folate targetd Par900 NPs and nontargeted Par788 NPs over time. Signals are coregistered with ultrasound, and blood signals. FIG. 15D is a graph showing spectrally unmixed nanoparticle signal quantification in a 3D tumor volume over time. Photoacoustic signal of regions in 3D regions of interest is mapped out and determined immediately after injenction, at t=24 hrs, and t=72 hours after inection. Multiple PAI NPs with different PAI activity and absorbance profiles can be imaged and co-registered in a single image over time. Images were taken with a Vevo LAZR Photoacoustic Imaging System. Greyscale=ultrasound, red=oxygentated. Note—color intensities between colors in images are not directly comparable to concentrations of each different component in mouse tumors. simultaneously imaging a tumor using two photoacoustic NPs at different wavelengths over time.

### Example 10

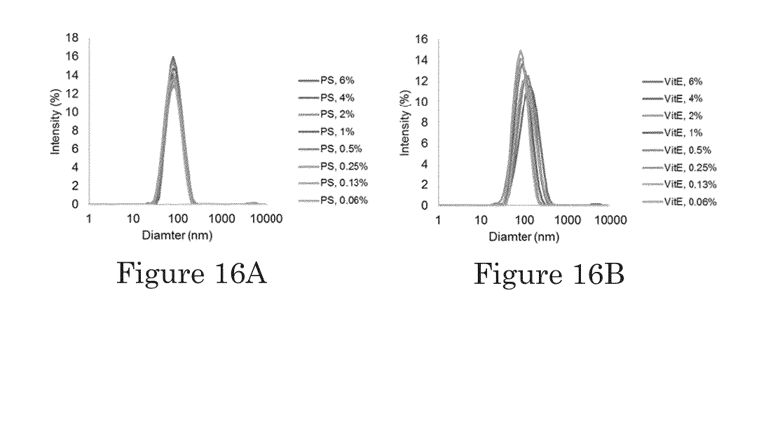

FIGS. 16A and 16B are graphs showing the formation of bacteriochlorin nanoparticles. The dye B56 was obtained from NIRvana Sciences, Raleigh, N.C. Dynamic light scattering size distribution spectra of PAI nanoparticles formed using B56, PS_{1.6k}-b-PEG₅ₖ as the amphiphilic stabilizer, and two different hydrophobic core materials: FIG. 16A shows 1.8 kDa polystyrene homopolymer. FIG. 16B shows alpha-tocopherol and B56 in THF at varying % B56 load and rapidly mixed with against water using a confined impingement jet. PAI particles are formed with polystyrene core with varying % B56 core weight particles (PS, B56%), or are formed with alpha-tocopherol core with varying % B56 core weight particles (VitE, B56%). NPs particles with either a polystyrene of alpha-tocopherol core, and at varying B56 loadings can be formed.

### Example 11

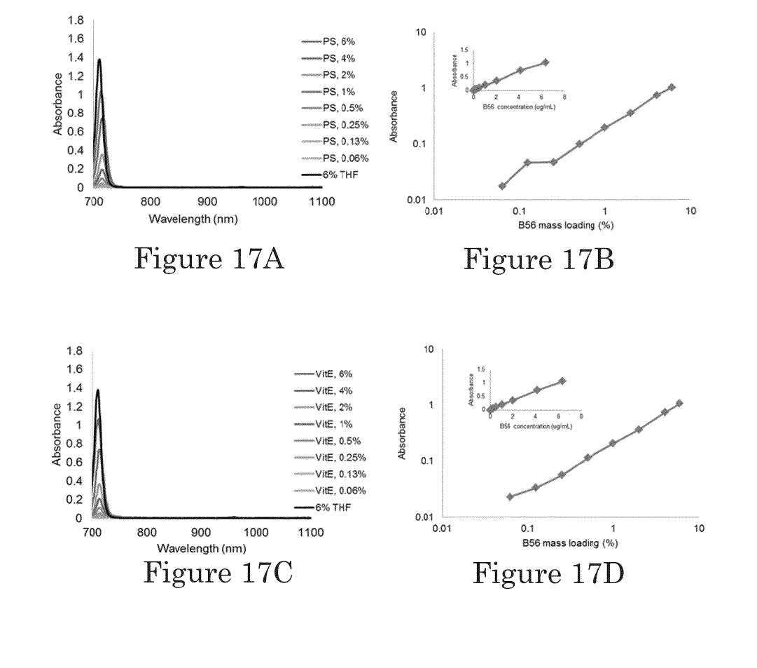

FIGS. 17A-17D are graphs showing the absorbance profiles of B56 NPs including: FIG. 17A—Absorbance spectra of B56 polystyrene core NPs with varying core B56 loading (PS, B56%), and of unencapsulated B56 dissolved in THF (6% THF); FIG. 17B—Absorbance of B56 polystyrene core NPs at 720 nm with varying particle core mass loading (VitE, B56%); FIG. 17C—Absorbance spectra of B56 alpha-tocopherol core NPs with varying B56 loading, and of B56 dissolved in THF; and FIG. 17D—Absorbance of B56 alpha-tocopherol core NPs at 720 nm with varying particle core mass loading. B56 NPs with a wide range of absorbance intensities can be formed. B56 NP constructs have a maximum peak absorbance within the 700-1100 nm wavelength window. All constructs have a low absorbance value that is below 15% of baseline absorbance within the 700-1100 nm wavelength window.

### Example 12

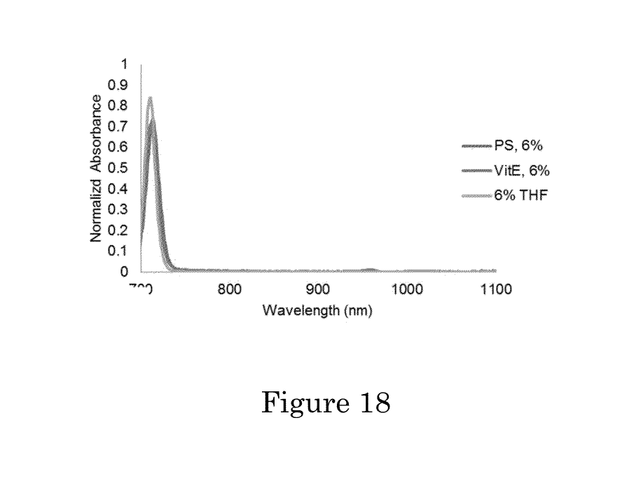

FIG. 18 shows the normalized absorbance profiles of B56 PAI NPs. Normalized absorbance spectra of B56 polystyrene core NPs with 6% core B56 loading (PS, 6%), B56 alpha-tocopherol core NPs with 6% core B56 loading (VitE, 6%), and of unencapsulated B56 dissolved in THF (6% THF). All constructs have a maximum peak absorbance within the 700-1100 nm wavelength window. All constructs have a low absorbance value that is below 15% of baseline absorbance within the 700-1100 nm wavelength window.

All references listed herein are also part of the application and are incorporated by reference in their entirety as if fully set forth herein.

### REFERENCES

1. Ryabenko, A., T. Dorofeeva, and G. Zvereva, UV-VIS-NIR spectroscopy study of sensitivity of single-wall carbon nanotubes to chemical processing and Van-der-Waals SWNT/SWNT interaction. Verification of the SWNT content measurements by absorption spectroscopy. Carbon, 2004. 42(8): p. 1523-1535.
2. Wang, C., et al., RGD-conjugated silica-coated gold nanorods on the surface of carbon nanotubes for targeted photoacoustic imaging of gastric cancer. Nanoscale Research Letters, 2014. 9(1): p. 1-10.
3. Taniguchi, M., et al., Accessing the near-infrared spectral region with stable, synthetic, wavelength-tunable bacteriochlorins. New Journal of Chemistry, 2008. 32(6): p. 947-958.
4. Xu, M. and L. V. Wang, Photoacoustic imaging in biomedicine. Review of scientific instruments, 2006. 77(4): p. 041101.
5. Luke, G. P., D. Yeager, and S. Y. Emelianov, Biomedical applications of photoacoustic imaging with exogenous contrast agents. Annals of biomedical engineering, 2012. 40(2): p. 422-437.
6. Razansky, D., C. Vinegoni, and V. Ntziachristos, Multispectral photoacoustic imaging of fluorochromes in small animals. Optics letters, 2007. 32(19): p. 2891-2893.
7. Tomida, Y., Photoacoustic imaging agent. 2008.
8. Wang, Y. and W. T. Shi, Photoacoustic imaging contrast agent and system for converting optical energy to in-band acoustic emission. 2009.
9. Wang, Y. and W. T. Shi, Photoacoustic contrast agent based active ultrasound imaging. 2012.
10. Sharma, P., et al., Multimodal nanoparticles for non-invasive bio-imaging. 2009.
11. Agarwal, A., et al., Targeted gold nanorod contrast agent for prostate cancer detection by photoacoustic imaging. Journal of applied physics, 2007. 102(6): p. 064701.
12. Li, P.-C., et al., In vivo photoacoustic molecular imaging with simultaneous multiple selective targeting using antibody-conjugated gold nanorods. Optics Express, 2008. 16(23): p. 18605-18615.
13. De La Zerda, A., et al., Carbon nanotubes as photoacoustic molecular imaging agents in living mice. Nature nanotechnology, 2008. 3(9): p. 557-562.
14. Yamauchi, F., et al., Contrast agent for photoacoustic imaging and photoacoustic imaging method using the same. 2011.
15. Hahn, M. A., et al., Nanoparticles as contrast agents for in-vivo bioimaging: current status and future perspectives. Analytical and bioanalytical chemistry, 2011. 399(1): p. 3-27.
16. Mayer, L. D., et al., Particulate constructs for release of active agents. 2005.
17. Green, F. J., The Sigma-Aldrich handbook of stains, dyes, and indicators. 1990: Aldrich Chemical Co.
18. Prud'homme, R. K., M. Gindy, and Y. Liu, Composite Flash-Precipitated Nanoparticles. 2008.
19. Liu, Y., et al., Mixing in a multi-inlet vortex mixer (MIVM) for flash nano-precipitation. Chemical Engineering Science, 2008. 63(11): p. 2829-2842.
20. Troiano, G., M. Figa, and A. Sabnis, Drug loaded polymeric nanoparticles and methods of making and using same. 2013.
21. Payne, M. M., S. R. Parkin, and J. E. Anthony, Functionalized higher acenes: hexacene and heptacene. Journal of the American Chemical Society, 2005. 127(22): p. 8028-8029.
22. Wolak, M. A., et al., High Performance Organic Light-Emitting Diodes Based on Dioxolane-Substituted Pentacene Derivatives. Advanced Functional Materials, 2006. 16(15): p. 1943-1949.
23. Pinkerton, N. M., et al., Formation of Stable Nanocarriers by in Situ Ion
Pairing during Block-Copolymer-Directed Rapid Precipitation. Molecular Pharmaceutics, 2012. 10(1): p. 319-328.

What is claimed is:

1. Nanoparticle constructs for photoacoustic imaging comprising:
a. a photoacoustic imaging agent (PAI) agent which has an absorbance maximum or plateau in the range of wavelengths 700-1100 nm, and which has a low absorbance value that is within 15% of baseline absorbance in this range,
b. the PAI agent is an organic compound;
c. the PAI agent has a solubility of less than 0.1 wt % in DI water at 20 C,
d. the nanoparticle has a size range from 20 nm to 1500 nm,
e. the nanoparticle has a core and a polymeric surface stabilizing layer,
f. the PAI agent is encapsulated in the core of the nanoparticle.

2. The nanoparticles of claim 1 wherein the nanoparticles are formed with sizes from 20 nm to 400 nm.

3. The nanoparticles in claim 1 further comprising excipients or therapeutic agents.

4. The nanoparticles of claim 1 further comprising an amphiphilic stabilizer.

5. The nanoparticles of claim 1 further comprising targeting ligands on the stabilizer to enable targeting of the PAI nanoparticle.

6. The nanoparticles of claim 1 wherein the nanoparticles are formed by copolymer directed rapid precipitation.

7. The nanoparticles of claim 6 wherein the nanoparticles are formed by Flash NanoPrecipitation.

8. The nanoparticles of claim 1 are formed by emulsion processes followed by solvent stripping.

9. The nanoparticles of claim 1 wherein the PAI agent has a solubility of less than 0.05 wt % in DI water at 20 C.

10. The nanoparticles of claim 1 wherein the PAI agent has a solubility of less than 0.01 wt % in DI water at 20 C.

10. The nanoparticles of claim 1 wherein the PAI agent has a desired low solubility by virtue of ion pair formation.

11. The nanoparticles of claim 1 wherein the PAI agent has a desired low solubility by virtue of covalent conjugation.

12. The nanoparticles of claim 1 wherein the PAI agent is at least one of bacteriochlorin, chlorin, isobacteriochlorin and porphyrin.

13. The nanoparticles of claim 1 wherein the PAI agent is a hexacene-based dye.

14. The nanoparticles of claim 1 wherein the PAI agent comprises dyes available under the trade designations Lumogen IR 765 and 788.

15. The nanoparticles of claim 1 wherein the PAI agent is a perylene and naphthyl dyes.

16. The nanoparticles of claim 1 wherein the PAI agent is a phthalocyanine based dye.

17. The nanoparticles of claim 1 wherein the PAI agent is a naphthalocyanine based dye.

18. The nanoparticles of claim 1 wherein the PAI agent is a dye available from Persis Science Inc. under the trade designation Par765, Par788, Par830, and Par900.

19. The nanoparticles of claim 1 wherein the organic compound further comprises a coordinated metal atom

20. The nanoparticles of claim 1 which are useful for diagnostic imaging.

21. The nanoparticles of claim 1 which are useful for animal imaging.
